(19) United States
(12) Patent Application Publication
(10) Pub. No.: US 2024/0008801 A1
(43) Pub. Date: Jan. 11, 2024

(54) SYSTEM AND METHOD FOR VIRTUAL PANCREATOGRAPHY PIPEPLINE

(71) Applicant: The Research Foundation for the SUNY, Albany, NY (US)

(72) Inventors: Arie Kaufman, Plainview, NY (US); Konstantin Dmietriev, Port Jefferson, NY (US); Shreeraj Jadhav, Stony Brook, NY (US)

(73) Assignee: The Research Foundation for the SUNY, Albany, NY (US)

(21) Appl. No.: 18/023,927

(22) PCT Filed: Sep. 1, 2021
(86) PCT No.: PCT/US2021/048632

§ 371 Date: Feb. 28, 2023

Related U.S. Application Data
(60) Provisional application No. 63/073,122, filed on Sep. 1, 2020.

(51) Int. Cl. A61B 5/00; G16H 10/60; G06T 7/00; G06T 7/11

(52) U.S. Cl. CPC A61B 5/425; A61B 5/7267; A61B 5/743; G16H 10/60; G06T 7/0012; G06T 7/11; G06T 2207/10081; G06T 2207/20081; G06T 2207/20084; G06T 2207/30096; G06T 2207/30092

(57) ABSTRACT

Systems and methods for virtual pancreatography (VP) are disclosed for non-invasive diagnosis and classification of pancreatic lesions. VP is an end-to-end visual diagnosis system that includes: automatic segmentation of the pancreatic gland and the lesions, extraction of the primary pancreatic duct, automatic classification of lesions into a plurality of lesion types, and specialized 3D and 2D exploratory visualizations of the pancreas, lesions and surrounding anatomy. Volume rendering is combined with pancreas and lesion centric visualizations and measurements for effective diagnosis.

### CROSS-REFERENCE TO RELATED APPLICATIONS

This application relates to and claims priority from U.S. Patent Application No. 63/073,122 filed on Sep. 1, 2020, the entire disclosure of which is incorporated herein by reference

### FIELD

The present disclosure is generally related to systems and methods the generation and display of three-dimensional visualizations of the pancreas for computer aided diagnoses and treatment planning.

### BACKGROUND

Pancreatic cancer (PC) is among the most aggressive cancers with less than 10% survival rate over a 5-year period. Such low rate is partially attributed to the asymptomatic nature of the disease, leading to most cases remaining undetected until late stage. Early detection and characterization of distinctive precursor lesions can significantly improve the prognosis of PC. However, accurate characterization of lesions on CT scans is challenging since the relevant morphological and shape features are not clearly visible in conventional 2D views. These features also overlap between lesion types making a manual assessment subject to human errors and variability in diagnosis: the diagnostic accuracy of experienced radiologists is within 67-70% range.

The conventional art provides inadequate solutions to imaging and diagnosing disease in the pancreas. Pancreatography, for example, requires an invasive endoscopic procedure to diagnose the pancreas. Conventional 2D imaging of the pancreas is also inadequate because, for example, the relationship between the lesions and pancreatic ducts is difficult to establish. Technological progress in image acquisition, processing, and visualization has enabled fundamentally new medical virtual diagnosis and virtual endoscopy For instance, virtual colonoscopy (VC) is one technique for evaluation of colorectal cancer that are highly effective using advanced visualization methods, such as electronic biopsy, colon flattening, and synchronized display of two (e.g., prone, supine) scans. Similarly, virtual endoscopy has been applied to bronchoscopy, angioscopy, and sinus cavity. Prostate cancer visualization and mammography screening are examples where rendering and machine learning techniques have assisted clinicians in decision-making. Specialized visualizations have also been used in surgical and therapy planning. 3D visualization was utilized in contrast enhanced rat pancreas, and MR microscopy for micro scale features in mouse pancreas. However, there are currently no comprehensive visual diagnosis systems for non-invasive screening and analysis of pancreatic lesions in humans.

There is thus a need for a designated system with tools and 3D visualizations developed specifically for the detection, segmentation and analysis of the pancreas and lesions, which are inherently 3D, in order to, for example, provide adequate diagnostic insights.

### SUMMARY

These and many more advantages are provided in the exemplary embodiments of the present systems and methods.

Embodiments of the present system and methods described herein provide a system and method for virtual pancreatography (VP). The system and method is used for the pancreatic cancer screening in radiological images, including but not limited to Computed Tomography (“CT”) and Magnetic Resonance Imaging (“MRI”).

Specifically, the system and method provides a set of tools to perform an automatic segmentation of the pancreas gland, pancreatic lesion(s), a physician-guided segmentation of the pancreatic duct, automatic holistic analysis of the entire input image, automatic histopathological classification of the pancreatic lesions and the corresponding grade of dysplasia, and a comprehensive 3D and 2D visualization tool. The system and method is a non-invasive diagnostic device intended for use in the diagnostic evaluation by a physician of patients with potential or confirmed pancreatic lesions.

The system and method described herein provides non-invasive pancreatic cancer screening in abdominal scans, such as conventional Computed Tomography (CT), spectral CT a.k.a. dual-energy CT (DECT), or magnetic resonance imaging (MRI), which is an enhanced and expanded Virtual Pancreatography (VP) system. The system and method provides functionality for automatic detection and segmentation of the pancreas gland, pancreatic lesions and the pancreatic duct (and alternatively a physician-guided segmentation of the pancreatic duct), automatic holistic analysis of the entire abdominal scan and all of the present abnormalities, classification of the present pancreatic lesion(s) into histopathological types (e.g., intraductal papillary mucinous neoplasm (IPMN), mucinous cystic neoplasm (MCN), serous cystadenoma (SCA), solid pseudopapillary neoplasms (SPN), and others) and the grade of dysplasia (e.g., low, intermediate, high), and a comprehensive visualization interface with a 3D rendering, tools and measurement capabilities.

Specifically, the system and method includes the following components:

Pancreas, lesion and duct detection and segmentation: The system and method preferably includes a segmentation module to perform an automatic detection and segmentation of the pancreas gland and pancreatic lesion(s). The segmentation module includes a neural network, which analyzes the input abdominal scan, detects the target structures, and generates a probability map for voxels that constitutes the target structures (e.g. pancreas gland, lesions), if present. Moreover, the segmentation module need not be limited to the pancreas and can be configured for the segmentation of other abdominal structures (e.g., liver, spleen, and others).

Interactive duct segmentation: the system and method preferably provides an extraction module that provides a radiologist (or other use, e.g. physician) an interface to segment the duct structures internal to the pancreas gland in a semi-automatic manner. This includes primary and secondary ducts as well as the bile duct, if visible. The extraction module starts with a denoising filter followed by extraction of local multi-scale cylindrical geometry, and connected component analysis to select significant vessels.

Holistic analysis of the abdominal scan: the system and method analyzes the entire abdominal scan to obtain a holistic picture of all of the clinical findings (e.g., spread of cancer, hepatic lesions, and others).

Lesion classification and prediction of the grade of dysplasia: Leveraging on the demographics of a patient and obtained holistic picture of the abdominal region, the classification module of the system and method performs an automatic histopathological classification of the pancreatic lesion(s) (if present) and the corresponding grade of dysplasia. The classification module includes two principal parts: (1) a probabilistic random forest classifier, which the age and gender of a patient, location of the lesion within the pancreas gland, its shape and intensity characteristics, derived from the outlines and the intensities of the scan; (2) a neural network, which analyzes the high-level imaging characteristics of the lesion(s). These two parts are combined into a Bayesian combination to encode the relationship between the estimated characteristics of the lesion(s) and its histopathological type, to, thus, produce the final classification probabilities. Another embodiment is when the machine learning of the segmentation is combined with that of the classification within the same deep learning network. Certain lesion types can be characterized, at least in part, by a proximity to the duct structure. Preferably, the system and method provides duct centerline information which can be input to the lesion classification module as a further input.

Visualization interface: The visualization module of the system and method constructs and renders enhanced 3D visualizations and 2D reformations of the pancreas, lesions, ducts, and surrounding anatomy. It includes a user interface (UI) with multiple linked and synchronized 3D and 2D viewports to support interactive visual diagnosis of the pancreatic lesions. It aids the physician in visual inspection of structural features from the scan that are relevant to classification and diagnosis of pancreatic lesions. This includes enhancement and 3D visualization of the entire scan and segmented structures such as the pancreas gland, lesions, and duct structures in different combinations; 3D visualization of internal lesion features such as septation, calcifications, cystic, and solid components; 3D visualization of pancreatic duct structures and its relationship/communication with the lesion; 2D multi-planar (axial, coronal, sagittal, and arbitrary planes) and curved-planar reformations (constructed through computed centerlines of the duct and the pancreas) for closer and time efficient analysis of duct-lesion relationship. The interface also provides automatic volume measurements of segmented lesion, pancreas, and duct volumes; automatic extent measures of lesions; and arbitrary interactive linear measurements in 3D and 2D viewpoints.

Multi-modal analysis: Multiple phases of the DECT such as the scans from multiple energy levels and algorithmically constructed single energy and material decomposition images such as iodine, fat, and water are utilized for providing comparitive visualizations (side by- side and overlaid comparison) as well as to further enhance 3D visualizations. Higher contrast between regions as well as additional edge and boundary information from these phases are algorithmically augmented for improved accuracy of segmentation, histopathological classification, and 3D/2D visualization of pancreas, lesions, and the ducts. In particular, DECT is utilized for better accuracy in detection of smaller (early) lesions and duct segmentation. When DECT is available, d1e pancreatic duct can be detected and segmented automatically.

The system and method assists the physicians (typically a radiologist) to improve the accuracy and speed of diagnosis and to ameliorate the objectivity of the differentiation of various pancreatic lesions identified in a radiological scan, such as CT (dual- or single-energy) or MRI. The system and method also supports early detection of pancretic lesions, which substantially changes the survival rates for pancreatic cancer.

### BRIEF DESCRIPTION OF THE DRAWINGS

For a more complete understanding of the present systems and methods, the objects and advantages thereof reference is now made to the following descriptions taken in connection with the accompanying drawings in which:

FIG. 1 illustrates various medical imaging of 2D slice views and 3D visualizations of a pancreas.

FIG. 2A depicts an illustration of a pancreas.

FIG. 2B depicts illustrative CT scans containing four exemplary types of pancreatic lesions.

FIG. 3A is a diagram illustrating an exemplary VP system.

FIG. 3B is a block diagram illustrating an exemplary process of the VP system.

FIG. 4 depicts a block diagram illustrating an exemplary architecture of the segmentation module.

FIG. 5 depicts various views of the pancreas and duct (views 5A, 5B, and 5C) using the segmentation, and duct extraction techniques of exemplary embodiments.

FIG. 6 illustrates two different views illustrating determined duct centerlines with a first and second approach.

FIG. 7 contains two graphical representations of a pancreas centerline segments (view 7A and view 7B) generated from techniques described herein,

FIG. 8 depicts an exemplary process by the classification module utilizing a CAD algorithm to classify pancreatic cystic lesions.

FIG. 9 depicts an illustrative snapshot of a user interface of the visualization module generated in accordance with exemplary embodiments.

FIG. 10 depicts various 3D visualizations that are generated by the visualization module in exemplary embodiments.

FIG. 11 shows examples of 3D pancreas-centric and curved planar reconstruction (“CPR”) views for two patients with dilated pancreatic ducts.

FIG. 12 depicts segmentations generated from exemplary embodiments and from manual segmentation.

FIG. 13 depicts examples of DLR and EFR visualizations of lesions.

FIG. 14 shows a visualization of the classification results in the user interface of the visualization module.

FIG. 15 shows a block diagram of an exemplary embodiment of a system according to the present disclosure.

### DETAILED DESCRIPTION

The following detailed description of embodiments of the present systems and methods will be made in reference to the accompanying drawings. In describing the invention, explanation about related functions or constructions known in the art are omitted for the sake of clearness in understanding the concept of the invention to avoid obscuring the invention with unnecessary detail.

Exemplary embodiments provide non-invasive computer-implemented systems and methods for virtual pancreatography (VP) using radiological images, including but not limited to Computed Tomography and Magnetic Resonance Imaging.

Exemplary embodiments employ a comprehensive visualization system that automatically or semi-automatically segments the pancreas and pancreatic lesions, and classifies the segmented lesions being one of a plurality of lesion types. It incorporates tools for user-assisted extraction of the primary duct, and provides effective exploratory visualizations of the pancreas, lesions, and related features. VP combines 3D volume rendering and 2D visualizations constructed through multi-planar reformation (MPR) and curved planar reformation (CPR) to provide better mappings between 3D visualization and raw CT intensities. Such mappings allow radiologists (and/or other users) to effortlessly inspect and verify 3D regions of interest using familiar 2D CT reconstructions.

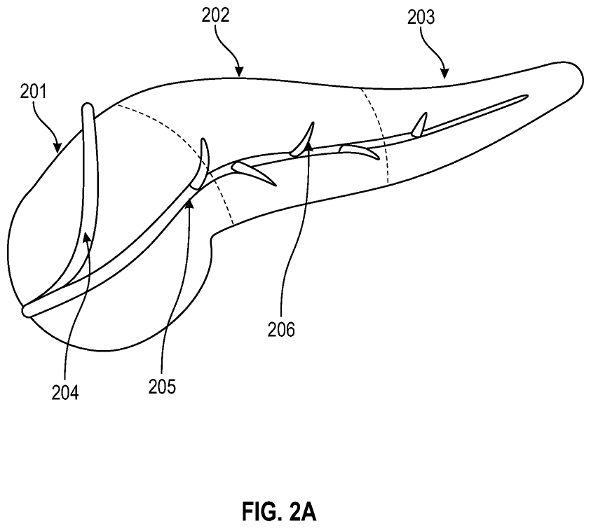

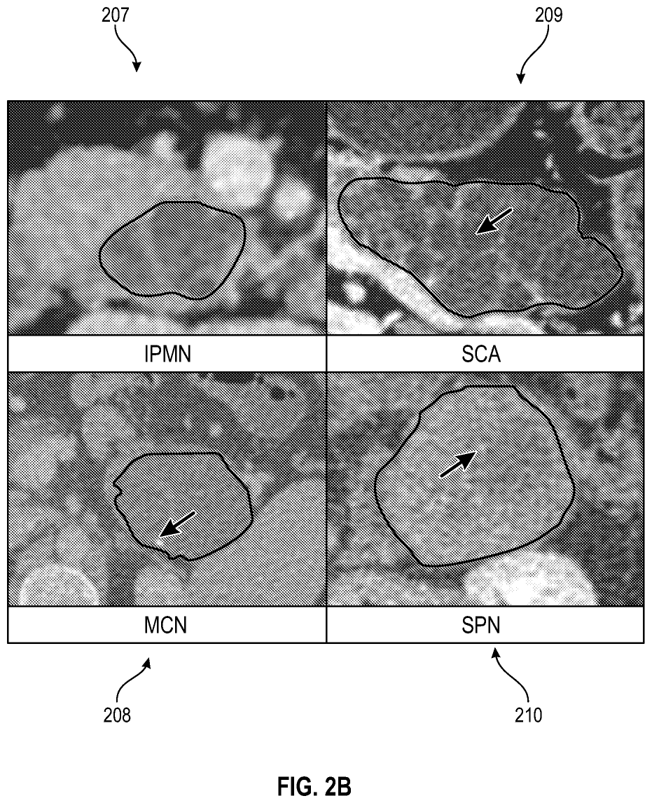

The pancreas is an elongated abdominal organ oriented horizontally towards the abdomen upper left side. It is surrounded by the stomach, spleen, liver, and intestine. It secretes important digestive juices for food assimilation and produces hormones for regulating blood sugar levels. As depicted in an illustration of the pancreas in FIG. 2A, the pancreas is broadly divided into three regions: head 201, body 202, and tail 203. The primary duct 205 passes approximately along the central axis of the elongated organ, and can be visible on a CT scan due to dilation. Also depicted in FIG. 2A is the bile duct 204, and a secondary duct 206. Pancreatic lesions are frequently encountered in CT scan and other types of medical imaging due to the increased usage of high-quality cross-sectional imaging. While weight loss, nausea, jaundice, and abdominal pain are common symptoms, the majority of cases are diagnosed incidentally. The most common types of pancreatic lesion are intraductal papillary mucinous neoplasm (IPMN), mucinous cystic neoplasm (MCN), serous cystadenoma (SCA), and solid-pseudopapillary neoplasm (SPN). These lesions vary in degrees of their malignancy and aggressiveness; IPMN and MCN are considered precursors to pancreatic cancer and offer the potential for early disease identification and intervention, whereas SCA and SPN have low malignancy potential.

The initial diagnosis of pancreatic lesions often starts with the age and gender of a patient, along with lesion location within the pancreas gland. The diagnosis is reinforced with the imaging characteristics identified on CT scans. Visible characteristics of lesions on CT images include features such as: (a) calcifications—calcium deposits that appear as bright high intensity specks; (b) cystic components—small (microcystic) and large (macrocystic) sacs typically filled with fluids that appear as dark approximately spherical regions; (c) septations—relatively brighter wall-like structures usually separating the cystic components; (d) solid components—solid light gray regions within the lesions; (e) duct dilation and communication—dilation of the primary duct and how it communicates with the lesion. The lesion morphological appearance based on these visible features can help in characterizing them. However, making a correct diagnosis is challenging since some diagnostic features overlap between different lesion types. Examples of the appearance of these lesions in CT scans are depicted in FIG. 2B. The main diagnostic features of these lesions are described below.

An IPMN is depicted in reference 207 in FIG. 2B. An IPMN is common in both males and females, typically with a mean age of 69, and can appear anywhere in the pancreas. Radiologically, IPMN has a microcystic appearance and communicates with the pancreatic duct. It typically does not have calcification.

A MCN is depicted in reference 209 in FIG. 2B. MCN is normally diagnosed in perimenopausal women with a mean age of 50 and is often located in the body or tail of the pancreas. It typically contains a single cystic component (macrocystic) and might have peripheral calcification and thick septation.

A SCA is depicted in reference 209 in FIG. 2B. SCA is frequent in women with a mean age of 65 and typically arises in the pancreatic head. CT appearance is usually a lobulated mass with a honeycomb-like (micro-or macro-cystic) form, which is often calcified.

A SPN is depicted in reference 210 in FIG. 2B. SPN mostly occurs in young women with a mean age of 25. The lesions illustrated in FIG. 2B typically appear as cystic components encapsulated by solid mass that may be calcified.

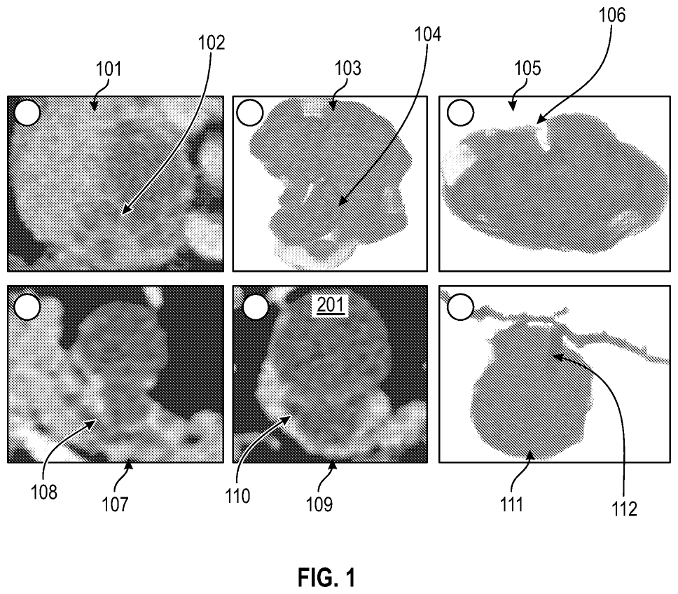

The VP systems and methods described in exemplary embodiments is superior to conventional 2D pancreatic imaging in diagnosis pancreatic disease. FIG. 1 illustrates various medical imaging of 2D slice views and 3D visualizations of a pancreas. 2D image 101 is an axial slice of a lesion on a pancreas head with an apparent internal lesion septation wall shown by reference 102. 3D visualizations 103 and 105 show the same region of the same pancreas as depicted in 2D image 101. As evidenced in 3D visualizations 103 and 105, however, the apparent internal lesion septation 102, is actually an external crevice (as pointed out in reference 104 and 106. Thus, 3D visualizations reveal important shape and size information of the lesion cystic components, which can impact the diagnosis. 2D image 107 in FIG. 1 is an image of an axial slice of a lesion and primary duct (the primary duct is pointed out by reference 108) in the pancreas body. In 2D image 109 secondary duct 110 that connects to the lesion appears very subtle on the slice view. 3D visualization 111, however, clearly shows the branching secondary duct 112 connecting with the lesion. Thus, 3D visualizations can draw attention to such subtleties.

Interpretation of lesion morphology on 2D views is often challenging. For example, as shown in reference numbers 101, 103 and 105 in FIG. 1, lesion walls with complex non-convex shape may appear similar to internal septation. Similarly, examining the duct is cumbersome as it splits into multiple components through cross sections as depicted in reference numbers 107 109 and 111 in FIG. 1. A typical span of the pancreas can be 150-200 axial slices. Mental reconstruction of 3D structure of lesion, duct, and duct-lesion relationship requires significant scrolling and mental effort. This can be both time consuming and error prone. Additionally, the variability in radiologists' expertise, lack of specialized tools for lesion detection, analysis, and accurate measurement, introduce diagnosis inconsistencies. Based on these time and quality challenges, we introduce specialized 3D tools.

The following non-exhaustive benefits and/or additional features are provided by exemplary embodiments herein. Employment of an automatic algorithm for pancreas and lesion segmentation. Providing 3D and 2D visualizations of the pancreas and lesions with inter-linked views to explore and acquire information from different views. The provision of preset modes to visualize the lesion and enhance its characteristic internal features. Deployment of a system and method for semi-automatic extraction of the pancreatic duct, which supports 3D visualization and 2D reformed viewpoints for detailed analysis of the relationship between the duct and lesions. The reformed viewpoints include (a) an orthogonal cutting plane that slides along the duct centerline (centerline-guided re-sectioning) to improve visual coherence in tracking the footprint (cross-section) of the duct across its length, and (b) a CPR of the pancreas to visualize the entire duct and the lesion in a single 2D viewpoint. Employment of simpler local 1D transfer functions (TFs) by leveraging the already segmented structures, and the provision of a simplified interface for transfer function manipulation to keep the visualizations intuitive and avoid misinterpretation of data. While being more extensive, this tool performs similar to grayscale window/level adjustment commonly used by radiologists. Incorporating a module for the automatic classification of lesions, based on a detailed analysis of the demographic and, clinical data and radiological features derived from radiological images. Also, the provision of a tool for automatic and manual assessment of the size of the lesions.

Exemplary embodiments provide comprehensive visualization tools for qualitative analysis using radiologists' expertise in identifying characteristic malignant features, while augmenting the diagnostic process with automated quantitative analysis, such as automatic classification and measurements.

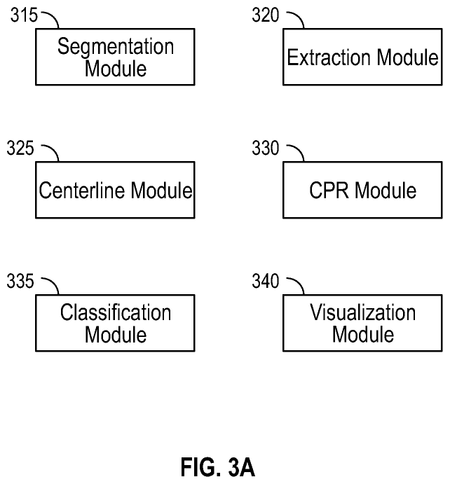

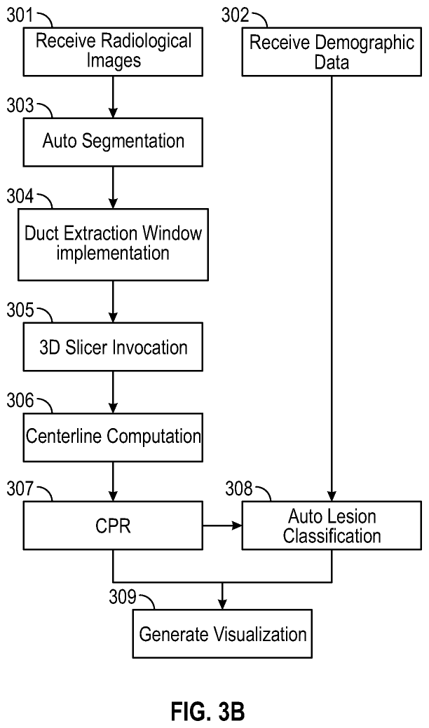

FIG. 3A is a simplified block diagram illustrating an exemplary VP system. Broadly, the exemplary VP system employs the following modules: (1) a segmentation module 315 to perform automatic and/or semi-automatic segmentation of the pancreas and lesions; (2) an extraction module 320 configured to facilitate the semi-automatic and/or automatic extraction of primary pancreatic duct, followed by centerline and CPR computation; (3) a centerline module 325 configured to compute the centerline of various segmented components of the pancreas; (4) a CPR module 300 configured to perform curved planar reformation of the segmented pancreatic components; (5) a classification module 330 configured to perform automatic lesion classification into a plurality (e.g., four) of common lesion types such as illustrated in block 307 in FIG. 3B; and (6) a visualization module 335 configured to provide a full featured user-interface for exploratory visualizations and measurement tools illustrated in block 308.

FIG. 3B is a simplified flow/block diagram illustrating an exemplary process of the VP system.

At block 301, the exemplary VP system can receive as an input radiological images of an abdominal region. This image data may be CT, MRI, multi-modal or other suitable radiological image data. At block 302 the VP system can receive demographic and clinical data, which can be used by the classification module as described further herein. Clinical data can include radiological images of other patients, and other signs or symptoms of such patients, and demographic data can include personal characteristics of the specific patient relevant to pancreatic disease diagnosis (including the patient's age, sex, health habits, geography, comorbidities, etc.).

At block 303 radiological images of a patient's abdominal region are first passed through the segmentation module, which segments the pancreas and pancreatic lesions, if present.

At block 304, segmentations created in the automatic segmentation module can be passed to the extraction module. The extraction module can include a graphical user interface that provides a duct extraction window that provides a radiologist or other technician, for example, with the ability to isolate the primary pancreatic duct interactively through a semi-automatic process. The step associated with block 304 is optional, and can be skipped if a duct is not visible in the radiological images.

At block 305, a radiologist or other technician, for example, can manually correct any errors in the segmentation masks through automatic invocation of 3D Slicer via the extraction module. Step 305 is also optional depending on any errors found during the segmentation process.

At block 306, once the segmentation results are finalized, the pancreas and duct centerlines can be computed by the centerline module.

At block 307 CPR of the pancreatic structures can be performed by the CPR module.

At block 308 the classification module performs automatic classification of the segmented lesions using inputted demographic and clinical data (from block 302), image data and centerline data from the segmentation results (from block 305). Preferably, the automatic lesion classification block generates a probabilities for a plurality of lesion types to assist in computer aided diagnostics of identified lesions. The CT volume, segmented pancreas, lesions, duct, computed centerlines, and the classification probabilities are then loaded into the main visualization module at block 309 to display visualizations to the user via a graphical user interface.

Segmentation Module. Being deeply seated in the retroperitoneum, the pancreas is quite difficult to visualize. Its close proximity to obstructing surrounding organs (stomach, spleen, liver, intestine), further complicates its visual analysis and diagnosis. Segmentation of important components (pancreas, lesions, duct) can provide better control on visualization design and interactions due to explicit knowledge of structure boundaries.

Exemplary embodiments employ an automatic segmentation process with high computational efficiency and universality via the segmentation module. The same architecture and training procedure to perform segmentations for both the pancreas and lesions can be utilized, demonstrating the generality of the embodiments disclosed herein. Additionally, the automatic segmentation process disclosed herein simplifies implementation because a common pipeline for both types of radiological images can be utilized. The architecture of the segmentation module is based on a multi-scale 3D ConvNet for processing 3D sub-volumes, which has two branches: (1) to detect whether or not the sub-volume contains the target structure for segmentation (healthy tissue of the pancreatic gland or tissue of a lesion), and (2) to predict a set of voxels that constitutes a target structure. In addition to improving convergence, such a two-branch model speeds up the inference by avoiding predicting a full-resolution segmentation mask for a sub-volume if it does not contain the target structure, speeding up the inference process, on average, by 34.8%.

The input to segmentation module is a 3D sub-volume of the original radiological image data (e.g. CT scan), and the outputs are a binary label and a binary mask of the target segmentation tissue. The segmentation module consists of decoder and encoder paths. However, unlike conventional systems, the encoder and decoder paths of the segmentation module do not mirror each other in exemplary embodiments described herein.

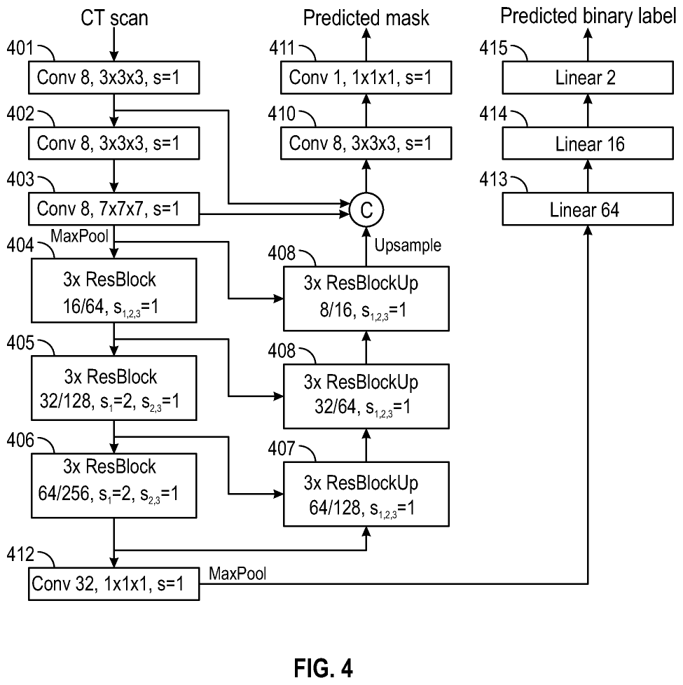

FIG. 4 depicts a block diagram illustrating an exemplary architecture of the segmentation module. The components depicted in FIG. 4 (including the encoder and decoder, the various kernel sizes and strides, and the number of layers, branches, and other functions) is merely illustrative and minor variations thereof, and other permutations are within the scope of the disclosure as will be apparent to one of ordinary skill in the art.

The encoder includes three consequently connected convolutional layers represented in blocks 401, 402, and 403, each of various kernel sizes and strides. Each convolution layer 401, 402, 403 is followed by a Leaky ReLU activation function and batch normalization layers, proceeded by three ResNet blocks represented in blocks 404, 405, and 406. The decoder includes three ResNet blocks represented by blocks 407, 408, and 409, followed by two convolutional layers represented by blocks 410 and 411, where the last layer 411 ends with a sigmoid function. Additionally, an auxiliary branch represented in block 412 is connected to the encoder to perform a binary classification of the input for the presence of the target structure. This auxiliary branch 412 is composed of one convolutional layer of 32 kernels of 1×1×1 size, followed by a max-pooling and reshaping layers and three fully-connected layers represented by blocks 413, 414, and 415, each connected by the Leaky ReLU function, while the last layer 415 ends with a sigmoid.

Advantageously, the segmentation architecture can be trained to minimize a joint loss, which is a summation of a binary voxel-wise cross-entropy from the auxiliary classifier and a Dice coefficient (DC)-based loss from the decoder part. However, the latter loss function can be challenging to optimize when the target occupies only a small portion of the input due to the learning process getting trapped in the spurious local minima with-out predicting any masks. Conventional approaches, for example, that target segmentation of the pancreas and pancreatic lesions, are inadequate because such approaches are tailored for segmenting a particular structure, and cannot be easily extended for other structures.

Exemplary embodiments on the other hand, advantageously minimize the DC-based loss function during segmentation for any structure, regardless of its size, using any base model. The optimization technique implemented in the segmentation module is based on the following iterative process. To alleviate the obstructive issues of the target structure being too small with regards to the overall input size, the segmentation module utilizes a process which trains a model on the smaller sub-volumes extracted from the center of the original sub-volumes and upsampled to the target input size. This allows a model to be pre-trained where the target structure can occupy a significant portion of the input sub-volume. By gradually increasing the size of the extracted sub-volumes, the model weights can be fine-tuned without the model getting stuck in a local minima. The model can be gradually trained in larger sub-volumes, and eventually, the model can be fine-tuned based on the original target resolution, which can be, for example, 32×256×256 in the case for CT scans involving both the pancreas and lesion segmentation.

Duct Extraction Module. The extraction module can facilitate a semi-automatic approach for duct segmentation, based on a multi-scale vesselness filter, using an adjustable threshold parameter. Vesselness filters can be designed to enhance vascular structures through estimation of local geometry. The extraction module can utilize the eigenvalues of a locally computed Hessian matrix to estimate vesselness (or cylindricity) of each voxel x∈P, where P is the segmented pancreas. Higher values indicate higher probability of a vessel at that voxel. Normalized vesselness response values R(x) can be computed thereafter. A user (such as a radiologist or other technician) can then adjust the threshold parameter to compute a response:

$$\\ \begin{matrix}{R}^{1} ( x ) = \{ \begin{matrix}0 , & R ( x ) < t \\ R ( x ) , & otherwise\end{matrix} & ( 1 )\end{matrix}$$

In certain embodiments, the foregoing process employs thresholding that results

in multiple connected components of R(x) if zero values are considered as empty space. Default value fort can be set to 0.0015, for example. A total vesselness value S_{Ci} can be calculated for each resulting connected component Ci as the sum of response values within Cᵢ:

S_{Ci}=Σ_{x∈Ci}R′(x) (2)

The pancreatic duct can be selected by the user (such as a radiologist or other technician implementing the graphical user interface provided via the extraction module) as the first n connected components with largest S_{Ci} values. In other words, the list of connected components is sorted by S_{Ci} values (largest value first), and the first n components are chosen as the duct segmentation mask. Integer value of n is chosen by user through a spin button. The default n=1 is sufficient in most cases. The use of the metric described in connection with Eq. 2 is more reliable than simply picking n largest components by volume, since it provides a more balanced composition of both size and vesselness probability of a component. While the vesselness enhancement filter is well-known, Eq. 2 can be applied to, for example, eliminate occlusion due to noisy regions and tailoring the filter to extract the pancreatic duct with minimal user interaction.

The extraction module provided herein significantly improves and simplifies the workflow in comparison to conventional manual or semi-automatic approaches. The duct segmentation techniques, in particular, is simpler than conventional approaches, and allows a radiologist, for example, to easily extract by merely adjusting a single slider.

Centerline Computation. Once the duct has been segmented and the Duct Extraction Window has been closed, the two centerlines: the duct centerline and pancreas centerline can be computed by the centerline module.

Both the pancreas and duct centerlines create navigation paths for viewing 3D rendering compared with orthogonal 2D raw CT sections of the pancreas volume. The duct centerline provides a strict re-sectioning along the duct volume, which can be used for closer inspection of duct boundary and cross-section in the raw CT data. It is also used for computing the CPR view. On the other hand, the pancreas centerline provides smoother re-sectioning of the entire pancreas volume as it is geometrically less twisted, and is also used by the automatic lesion classification module for determining the location of the lesion within the pancreas.

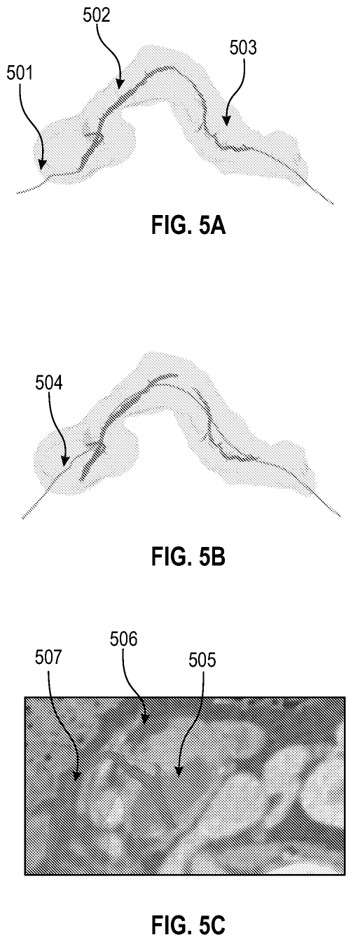

FIG. 5 depicts various views of the pancreas and duct (views 5A, 5B, and 5C) using the segmentation, and duct extraction techniques described herein.

As shown in view 5A in FIG. 5, duct centerline 501 through two components of primary duct 502 and 503 following the pancreas elongated shape, while approximately maintaining constant distance from the pancreas surface. View 5B shows pancreas centerline 504. View 5C shows a centerline-guided re-sectioning view embedded in the 3D view for the same pancreas shown in views 5A and 5C. Reference 505 shows the lesion volume, reference 596 shows the pancreas surface, and reference 507 shows the duct volume.

Pancreas Centerline. The pancreas centerline can be computed using a penalized distance algorithm. The penalized distance algorithm can, for example, utilize a graph to represent paths in a grid of voxels. Penalty values are assigned to every voxel (graph node) x in pancreas P, based on that voxel's distance from the pancreas surface. The path between the pancreas extreme ends with the minimum cost in the graph, can be calculated as the centerline. A distance field d(x) is computed on each node as the shortest distance to the pancreas surface. Because the centerline can be assumed to pass through the pancreas inner-most voxels, exemplary embodiments can assign higher penalty values to for voxels closer to the surface. Thus, each voxel distance can be subtraced from the maximum distance dₘₐₓ:

d′(x)=dₘₐₓ−d(x) 93)

This results in higher penalty values for the voxels closer to the pancreas surface, as desired. Note that d′(x) is always positive, as d(x)≤dₘₐₓ. The distance field d′ can be used as penalty values and compute the pancreas centerline.

Duct Centerline. Due to the noise in CT scans and variability in dilation, the extracted duct volume can be fragmented into multiple connected components. Since the pancreas itself is an elongated object, the duct centerline can be modeled to follow the entire pancreas length, as well as pass through every connected component of the extracted duct. The centerline follows the pancreas shape wherever the primary duct is disconnected. The penalized distance algorithm focuses on a single object boundary for computing skeleton curves. When computing the duct centerline, the problem of handling multiple objects (multiple connected components of the duct and pancreas) can arise. One first approach is to individually compute centerlines of duct components connected with one another other and to the pancreas extreme ends using the penalty field d′. However, such an approach can lead to excessive curve bending as the computed duct centerline enters and exits the duct components due to abrupt change in penalty field.

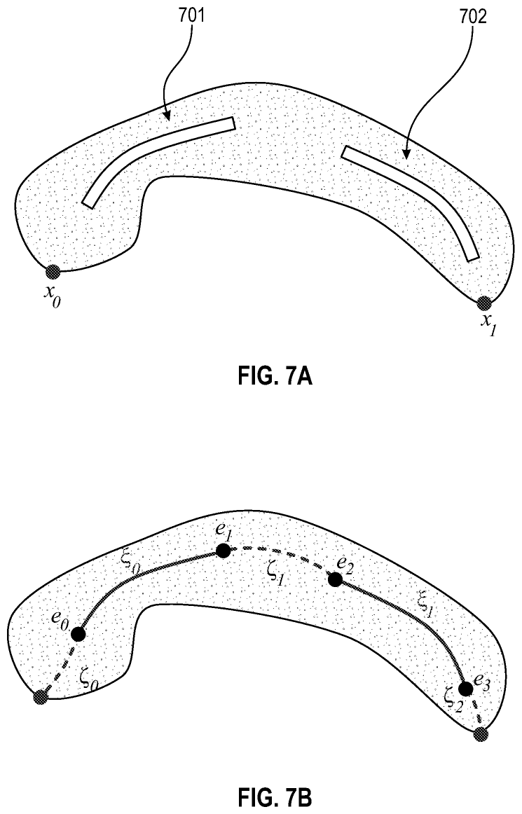

A second more advantageous approach is to model a curve that passes through the primary duct components and also respects the pancreas geometry without excessive correction as it enters and exits the duct components. The duct centerline can use the same end-points (x₀, x₁) as the pancreas centerline, and pass through all of the extracted duct fragments. In local regions where the primary duct is not contiguous, the duct centerline can follow the pancreas shape. This can be achieved by a trade-off between finding the shortest path and maintaining a constant distance to the pancreas surface. In one example, the trade-off is modelled through a modification of the penalized distances used in the algorithm as described below (see FIG. 7).

The method first calculates the centerlines {ξi} for each connected component of the duct volume independently, using the same process as described for the pancreas centerline. The centerline fragments are then oriented and sorted correctly to align along the length of the pancreas. Each pair of consecutive fragments (ξk, ξk+1) are then connected by computing the shortest penalized path {ζi} between their closest end-points. This includes connections to the global end points x0 and x1. When computing a connecting curve ζ0 between end points e₁ and e₂, the previously computed pancreas distance field d·(x) from Eq. 3 is transformed as:

d″(x)=|l−d′(x)| (4)

where l=(d(e₁)+d′(e₂))/2 is the average value between e1 and e2. The modified distance field d(x) is used as penalty values in the voxel graph for computing the connecting curve λ1. Together, alternating curves {ζi} and {ξi} form a single continuous duct centerline.

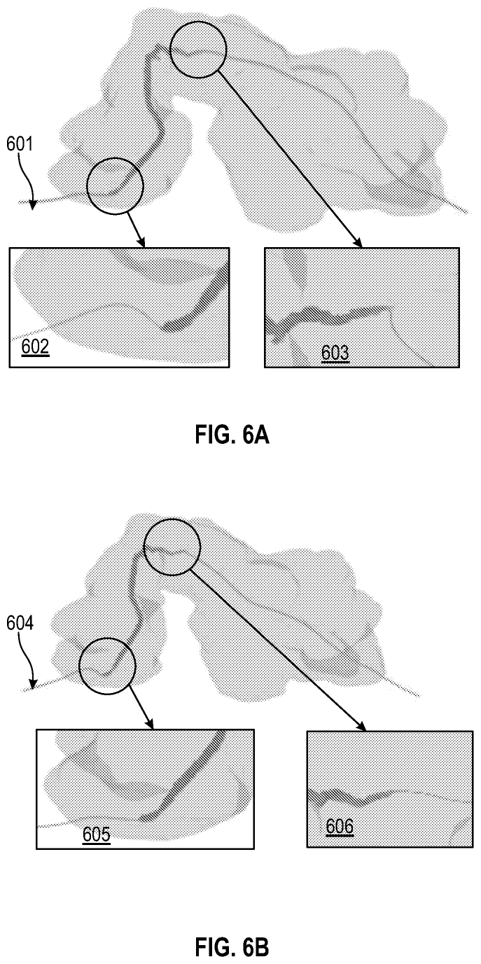

FIG. 7 contains two graphical representations of a pancreas (view 7A and view 7B) generated from techniques described herein, and illustrates the second approach for determining a duct centerline. View 7A shows a pancreas with two duct components (701 and 702). x0, x1 are end-points of pancreas centerline. View 7B shows ξ0 and ξ1, which are detached duct component centerlines. ζ0, ζ1, ζ2 are connecting curves. Together, the path ζ0, ξ0, ζ1, ξ1, and ζ2 forms the entire duct centerline. FIG. 6 illustrates two different views 6A and 6B illustrating determined duct

centerlines with the first and second approach, and highlights the benefits of using the second approach. View 6A illustrates a duct centerline 601 determined using the first approach (using penalty field) and view 6B illustrates a duct centerline 604 determined using the second approach. As shown in the comparison between the entry and exit points of the duct centerline into the primary duct (i.e. entry point 602 in approach one, compared to entry point 603 in approach two, and exit point 603 in approach one, compared to entry point 605 in approach two) shows a reduction in bending at entry and exit points in the second approach compared to the first approach. The results of the second approach are more effective as a smoother centerline is necessary for constructing CPR and re-sectioning views. CPR Module. A CPR module can utilize the segmented pancreatic components

and the computer centerlines of the components to create visualizations that can be loaded into the visualization module as described further herein with reference to the visualization module. The CPR module can be incorporated into the visualization module, or can be a distinct module. Classification Module. A CAD algorithm can be utilized by the classification

module for classification of pancreatic cystic lesions, that can use patient demographic and clinical information and the CT images as input. The classification module can, for example, comprise two main modules: (1) a probabilistic random forest (RF) classifier, trained on a set of manually selected features; and (2) a convolutional neural network (CNN) for analyzing the high-level radiological features. An example of such a classification module is described in international patent publication WO 2019/005722, the disclosure of which is incorporated by reference in its entirety. To automatically estimate the lesion location within the pancreas, the centerline of the pancreas can be divided into three even segments, representing its head, body, and tail, and the closest segment to the segmented lesion center of mass can be determined. The final classification probabilities for the four most common lesion types can be generated with, for example, a Bayesian combination of the RF and CNN, where the classifiers predictions can be weighted using the prior knowledge in a form of confusion matrices.

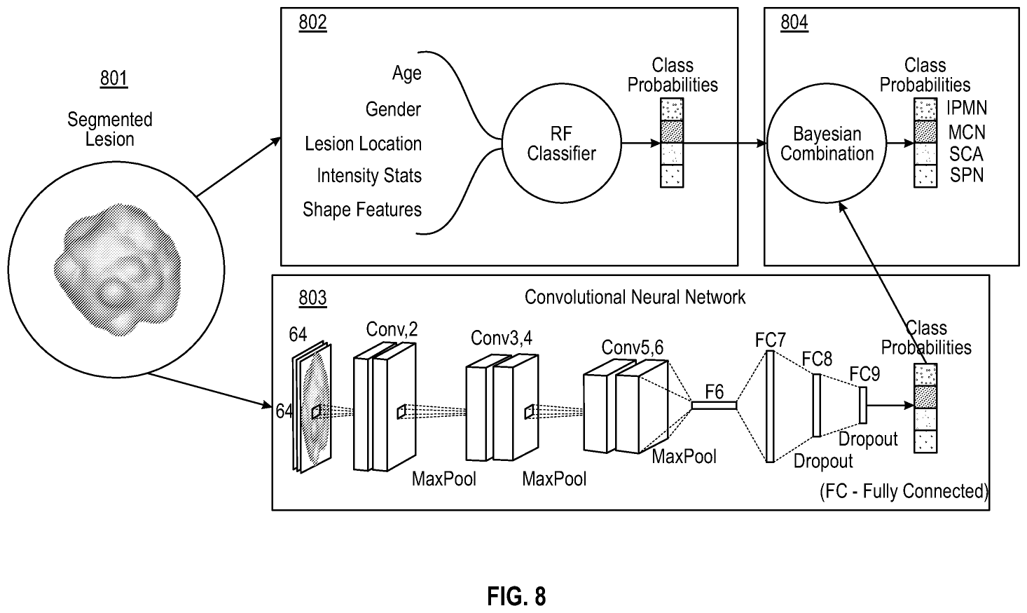

FIG. 8 depicts an exemplary process by the classification module utilizing a CAD algorithm to classify pancreatic cystic lesions. Reference 801 represents inputted segmented lesions which can be generated utilizing systems and methods described herein. Block 802 represents the probabilistic RF classifier, can be trained with a set of manually selected features from the demographic and clinical data. RF classifier can receive as an input the segmented lesion from 801. Block 803 represents a CNN for analyzing the high-level radiological features of the segmented lesion, which can be inputted into the CNN as well. The output of both the probalistic RF classifier 802 and CNN 803 can be combined via a bayesian model represented in block 804.

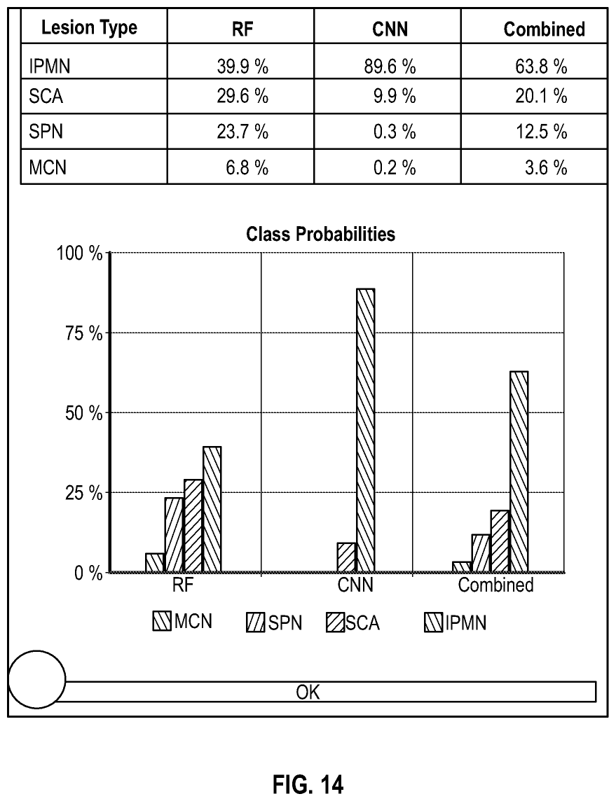

FIG. 14 shows an example of visualization of the classification results in the user interface of the visualization module.

The lesion classification system and methods described herein advantageously provide assistance during the diagnosis process. Viewing the RF and CNN classification probabilities separately before they are combined further allows additional control to weigh the importance of each and make further refinements for final diagnosis.

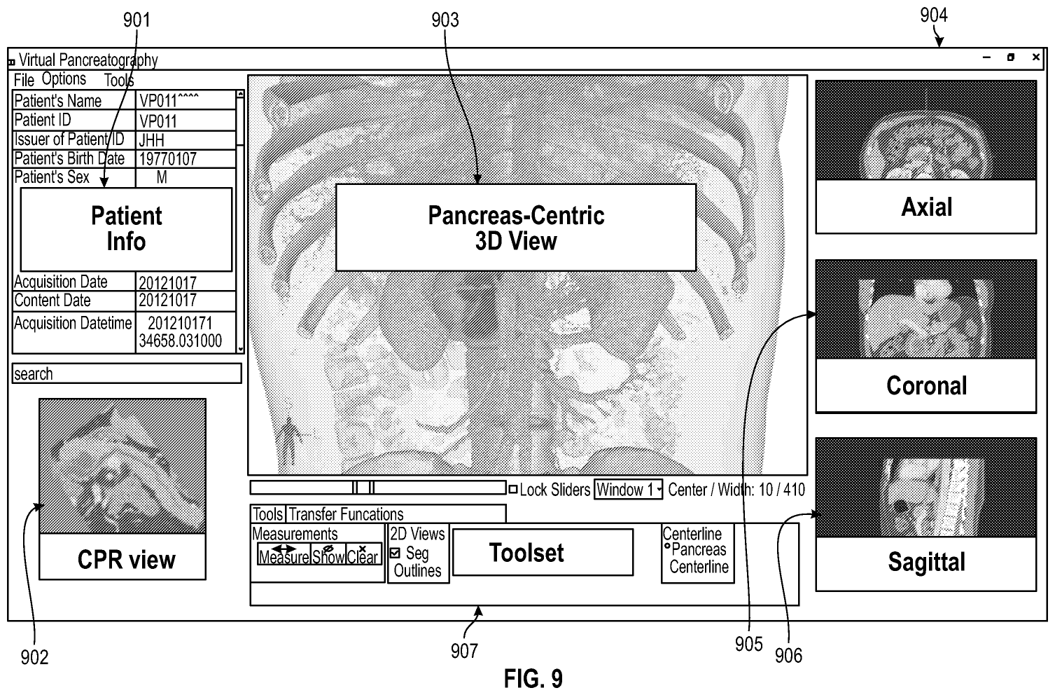

Visualization module. Once the pancreas, lesions, and duct are segmented, the centerlines are computed, and the classification probabilities are generated, they are available in a graphical user interface for visual diagnosis via the visualization module. An illustrative snapshot of a user interface of the visualization module generated in accordance with exemplary embodiments is shown in FIG. 9. The user interface can be divided into a plurality of frames to simultaneously provide corresponding 2D, 3D, demographic and diagnostic data to the user. In one embodiment, the graphical user interface provides seven viewing frames, although it will be appreciated that the specific configuration and number of the frames may be configured with more or less frames visible and different data selected to be displayed in each frame. Frame 901 in FIG. 9 can be a searchable table with the patient information and CT image metadata extracted from the CT Scan DICOM header. Below the patient information table, in frame 902 can display a pancreas CPR 2D view that can switch between Centerline-Guided Re-sectioning and Duct-Centric CPR view. In frame 903, a viewport displays the Pancreas-Centric 3D view for a plurality of 3D visualizations generated in exemplary embodiments herein. On the right hand side of the illustrative user interface, the three planar view frames: axial 904, coronal 905, and sagittal 906 can be displayed that are common in radiology applications. The three different planar view frames can support the interactions common in radiological interfaces, including panning, zooming, and window/level adjustment.

Linking of rendering canvases such as viewports and embedded cutting planes/surfaces is often used for correlation of features across viewpoints in diagnostic and visual exploration systems. In exemplary embodiments the 3D and 2D views are linked to facilitate correlation of relevant pancreas and lesion features across viewpoints. A point selected on any of the 2D views can automatically navigate the other 2D views to the clicked voxel. Additionally, a 3D cursor highlights the position of the selected voxel in the 3D viewpoint. Similarly, the user can also directly select a point in 3D by clicking from two different camera positions (perspectives). Each time the user clicks the 3D view, a ray is cast identifying a straight line passing through the 3D scene. Two such clicks uniquely identify a 3D point. The 2D views are automatically changed to the selected voxel. This linking of views allows comparison between features in 3D visualizations with CT raw intensities in 2D using the illustrative user interface.

At the screen bottom in FIG. 9 is toolset 907 that provides user tools/options to control the 3D and 2D visualizations and their parameters in the visualization module. These include, for example, visibility (show/hide) and opacity of segmented structures, clipping planes, and window/level setting (grayscale color map) common for all the 2D views.

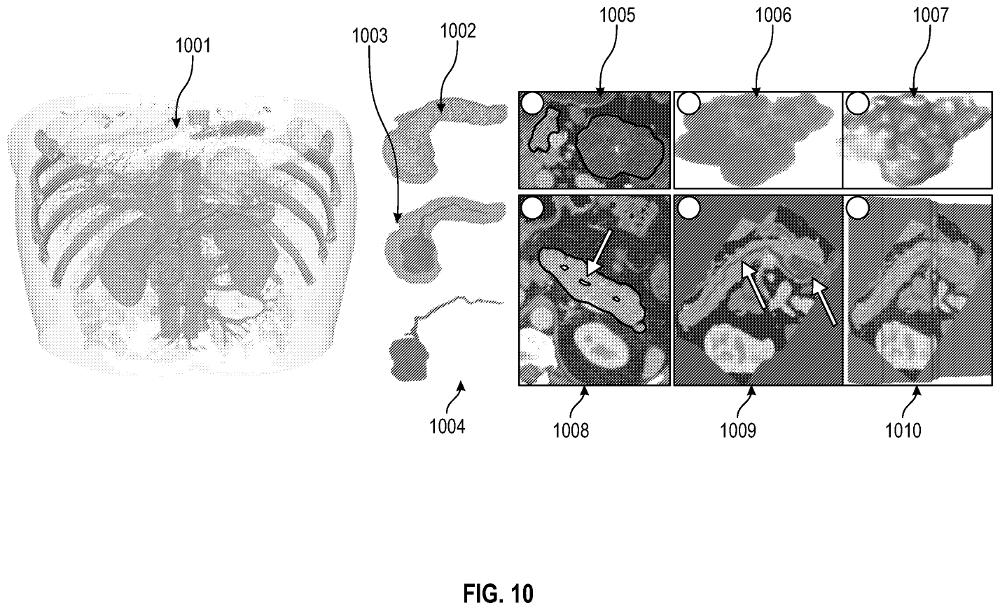

FIG. 10 depicts various 3D visualizations that are generated by the visualization module in exemplary embodiments. Various pancreatic components displayed in the visualization module (such as septations, cystic components, lesions, ducts, etc) can be displayed in different colors, with different textures and lighting effects, to make it easier for users to understand the visualizations and make proper diagnoses.

3D visualizations, 1001, 1002, 1003, and 1004 in FIG. 10 are examples of pancreas-centric 3D visualizations. 1001 includes context volume around the pancreas clipped from the top and bottom for focused visualization. Visualizations 1002, 1003, and 1004 include the segmented anatomical structures and the CT volume rendered in different combinations. Visualizations 1005, 1006, and 1007 are examples enhanced lesion visualizations. Visualization 1005 is an example of an axial view of a lesion and pancreas. Visualization 1006 is a direct lesion rendering (DLR) view showing septation and cystic components. Visualization 1007 is an enhanced features rendering (EFR) view showing calcifications and septation. The visualization 1007 can be, for example, enhanced through a Hessian-based objectness filter to provide clearer view of internal lesion features. Visualizations 1008, 1009 and 110 show CPR views of a pancreas. Visualization 1008 is an Axial 2D view of pancreas, where the pancreatic primary duct does not lie in a single plane and appears broken. Visualization 1009 shows a Duct-centric CPR view of the pancreas: where the primary duct is completely visible in a single plane along with a lesion sectional view. Visualization 1010 shows a CPR surface embedded in 3D view: incorporating such a view along with 3D visualization is helpful in understanding how the CPR view was constructed.

The Pancreas-Centric 3D view renders the segmented anatomical structures (pancreas, lesions, duct) individually or in different combinations in the user interface of visualization module, as shown in FIG. 10 references 1002, 1003 and 1004. The context around the pancreas can also be visualized in the form of the entire CT volume. The user interface can display two clipping planes (top and bottom) to restrict this context view (such as shown in 1001 in FIG. 10). This is useful to reduce occlusions and focus on the region of interest.

The transfer function can be pre-designed for the context volume (CT volume surrounding the pancreas) and may not, for example, require manual editing as CT intensities have a similar range across patients. Similarly, the TFs used for the pancreas, lesions, and duct volume can also be pre-designed and are re-scaled to the scalar intensity ranges within the segmented structures for every patient. Exemplary embodiments provide simplified controls over the optical properties of the 3D visualizations rendered. They can be modified through a simplified interface using two sliders: opacity and offset; rather than editing a multi-node polyline which can have significantly more degrees of freedom. The opacity slider can control the global opacity of the segmented structure and the offset slider can apply a negative/positive offset to the transfer function to control how the colors are applied to the segment. The user interface can also include an advanced tab in the toolset to facilitate the 1D TFs to be edited as a multi-node polyline, if desired.

The 3D visualizations generated and displayed by the visualization module as described herein have the following additional benefits. The 3D pancreas-centric visualizations that combine pancreas outline along with lesion and duct volumes are very useful to assess the duct and lesion relationship. For example, in case of an IPMN it is critical to investigate if the lesion arises from the duct. The 3D visualizations can also facilitate the ease in which the shape of duct dilation can be identified so that further analysis of the IPMN type (main duct vs side branch communication) can be facilitated. Such a feature not only helps in lesion characterization, but can also better inform the decision making in surgery compared to conventional techniques. Such analysis would be much harder in 2D planar views as the shape of duct dilation and connectivity with side branches can be misleading in 2D slices. Further, 3D visualization and measurement capabilities of the system can be useful for a pre-operative planning since the surgeons can visualize the structures in 3D before the actual procedure. In addition, the two-slider transfer function editing interface is very simple and intuitive as compared to conventional systems (e.g. editing a multi-node polyline).

As described in more detail herein, the morphology and appearance of the pancreatic lesions (such as those described in connection with FIG. 2) on CT scans are paramount in their diagnosis. This includes an assessment of visible characteristics of the lesions, such as septation, central and peripheral calcification, and presence of cystic and solid components. To this end, exemplary embodiments provides visualizations of the internal features of lesions in two different modes, namely: direct lesion rendering (DLR) and enhanced features rendering (EFR).

The DLR visualization mode can perform a lesion direct volume rendering using a two-color (red, yellow) preset transfer function that applies a higher opacity red color for darker regions (e.g., cystic components), and a lower opacity yellow color for relatively brighter ones (e.g., septation, solid components). Other colors, and shades thereof, can be substituted for the ones described herein and be within the scope of the disclosure. This allows the radiologist to easily find the correct boundary between features (e.g., septation, cystic components). Another example of DLR rendering is reference 1006 in FIG. 10.

To enhance features in volume rendering, the EFR mode visualizes the lesion through enhancement of local geometry using a Hessian-based enhancement filter (such visualizations are shown in references 1001, 1002, 1003, and 1004 in FIG. 10). A locally computed Hessian at multiple scales can quantify the probability of a local structure, such as a blob, plate, or a vessel. The plate enhancement can be applied for wall structures and blob enhancement along with thresholding for calcifications. Both volumes can then be combined as a 2-channel (red-green) 3D texture for simultaneous volume rendering of enhanced features (such as the visualization shown in reference 1007 in FIG. 10).

The 3D lesion visualization methods described herein have the following additional benefits. The septation, cystic components, and calcifications are more visible than they appear in conventional pancreatic imaging approaches. Further, it is easier to understand the relative sizes of the cystic components, particularly in cases of SCA and SPN that have macro-cystic appearance. In addition, it easier to characterize IPMN lesions through the lesion 3D shape, which is easily identifiable in the main duct and side branch IPMNs, since side branch IPMNs have a more regular spherical shape as compared to main duct IPMNs.

Exemplary embodiments incorporate an exploratory visualization that combines 3D visualization with a 2D slice view of raw CT intensities. An orthogonal intersecting plane slides along either the pancreas or duct centerline and renders the raw CT intensities in grayscale. An example of this is shown in reference 5C in FIG. 5. Simultaneously, all 3D anatomical structures (pancreas, lesions, primary duct, surrounding CT volume), can be combined in the visualization through visibility controls. The sliding plane represents a centerline-guided re-sectioning of the pancreas volume, and can be viewed in the 3D view and separately as a 2D slice view. This sectional view provides coherent tracking of the primary duct along its length and closely studying its relationship with the lesion. Radiologists or other users can take a closer look at how the 3D anatomical structures, such as lesions and duct, correspond with raw CT values by directly overlapping the 3D rendering with the intersecting plane. This approach instills greater confidence in the radiologists and other users during diagnosis as they can correlate 3D structures to 2D images that they are traditionally interpreting. It also provides a better comparison than switching context across multiple viewports.

Additional benefits associated with the centerline-guided re-sectioning view embedded in the 3D pancreas-centric view include providing the ability to verify 3D features by directly correlating them with the 2D intersection plane of raw CT intensities. Through implemented linked views and point selection, radiologists and other users can easily relate corresponding points between 3D and 2D views.

The primary duct size and its relationship with pancreatic lesions is often important for visual diagnosis. For example, IPMN lesions communicate with the primary duct whereas other lesion types typically do not. CPR techniques reconstruct the longitudinal cross-sections of vessels, which enables visualization of the entire vessel length in a single projected viewpoint. Points on the vessel centerline may be swept along an arbitrary direction to construct a developable surface along the vessel length. The surface samples the intersecting volume at every point and is flattened without distortion into a 2D image. Using the computed duct centerline as a guide curve, the pancreas duct-centric CPR views can be generated, such as those depicted in 1008, 1009, and 1010 in FIG. 10. The direction in which the centerline is swept CPR surface construction is chosen by the user through a widget that shows the surface rendering of the pancreas, lesion, and centerline curve. The sweeping direction is preferably normal to the screen. This widget allows the user to choose an appropriate direction to generate the desired CPR viewpoint, thus helping in visualizing the duct-lesion relationship in a single viewpoint.

Additional benefits of the CPR reconstruction processes described herein include providing the primary duct and lesion in a single view. Through the 3D embedded CPR view showing both the CPR surface and 3D rendering of the duct and lesion, a radiologist or other user can verify that the duct is connected to the lesion, and can also identify how the duct dilates as it connects to the lesion. The duct dilation geometry would not be clearly visible in 2D views. Visualizing this dilation can also help in surgical decisions such as the location of potential resection to remove the malignant mass.

Measurement is an integral part of visual diagnosis as it helps to quantify lesion size and cystic components. The visualization module advantageously provides users with the capability to interactively measuring in all our 2D planar views, by clicking and drawing measurements. The visualization module also provides automatic lesion 3D measurements using the dimensions of a best fitted oriented bounding box around the lesion. Additionally, automatic volume measurements of segmented structures (pancreas, lesions, duct) can also be available via the user interface.

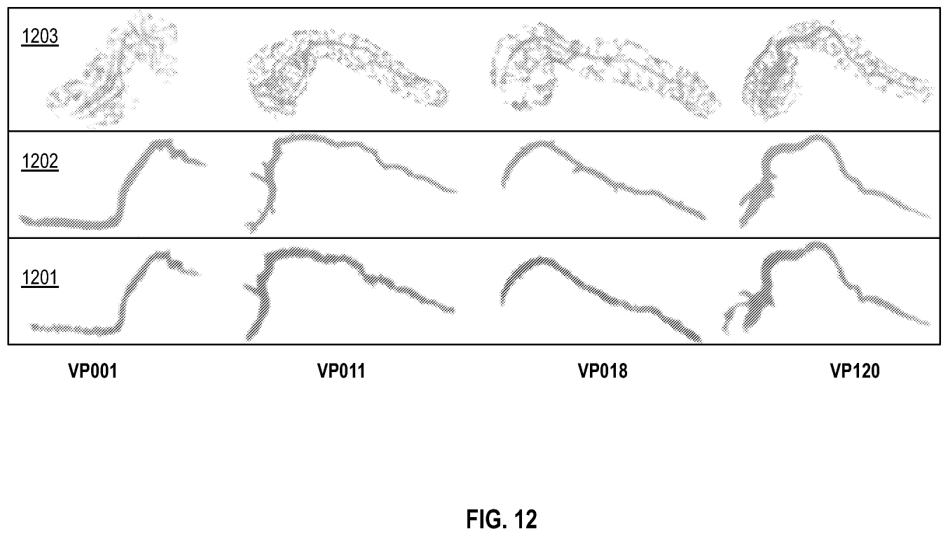

A qualitative comparison of the duct extraction approach described in exemplary embodiments with those generated from manual segmentations is shown in FIG. 12 with references 1202 and 1203. Exemplary duct and centerline extraction techniques described herein are able to overcome the noise and occlusions caused by noisy voxels that are mistakenly enhanced with the vesselness filter (shown on 1201). Additionally, the duct centerline approach described herein reduces the excessive bending as it enters and exists the duct by utilizing a penalty model which weights both duct and pancreas geometry when computing the curve (as described in connection to FIG. 6).

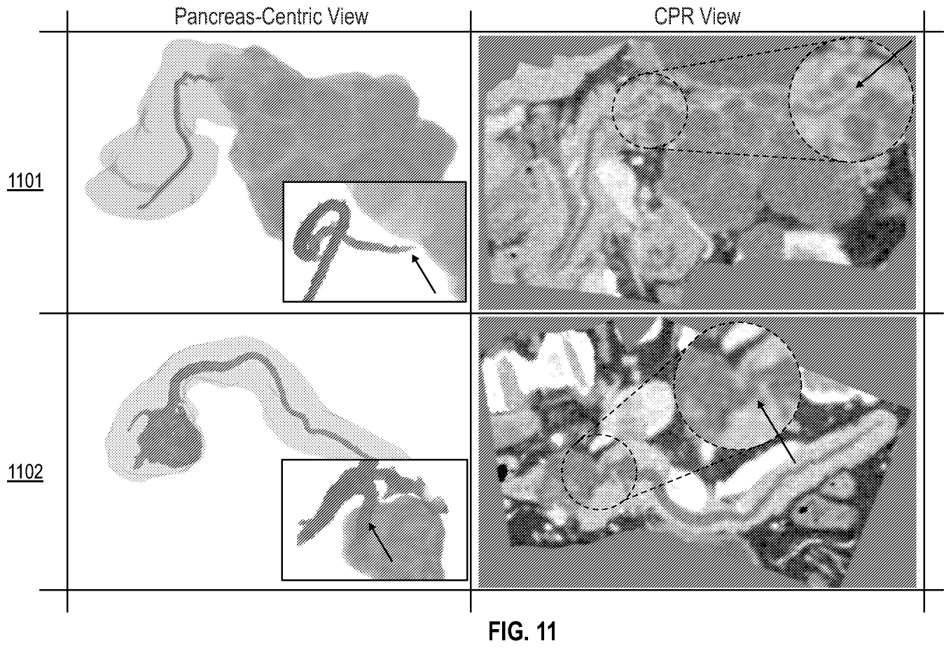

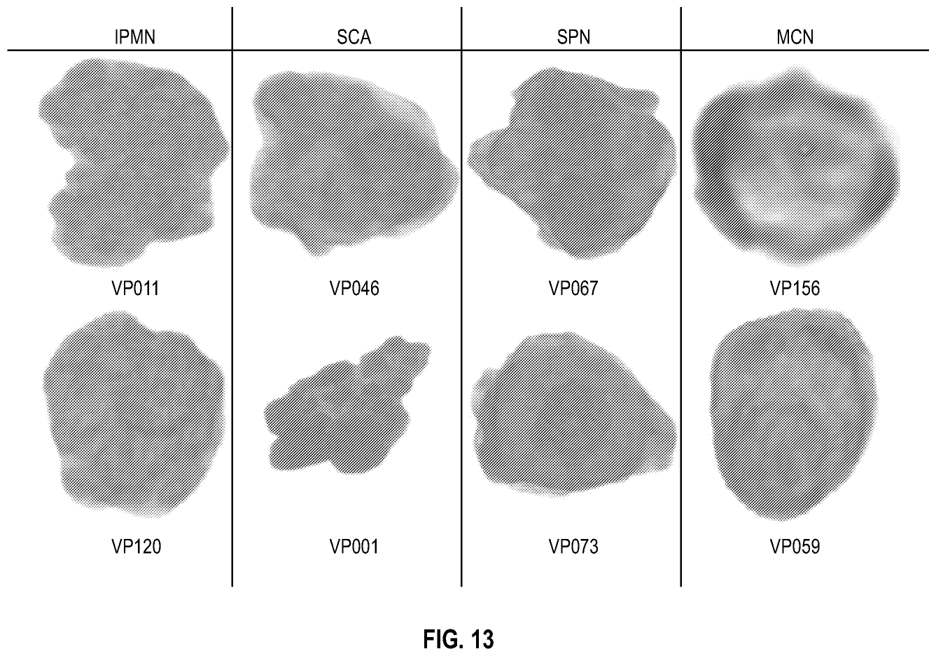

Examples of the 3D pancreas-centric and CPR views are shown in FIG. 11 for two patients with dilated ducts. These views can, for example assist in determining the presence of connection between the duct and the lesion in particularly difficult cases when the conventional 2D axial views are inadequate. Row 1101 in FIG. 11 confirms a presence of a duct-lesion connection, and row 1102 that confirms the absence of a duct-lesion connection. Examples of the DLR and EFR visualizations of the lesions are shown in FIG. 13. These visualizations can facilitate better understanding of lesions shape and structure in comparison to viewing them in 2D images, by enhancing the characteristic lesion structures, such as external wall, calcification, internal septation, solid and cystic components.

In the conventional workflow, radiologists generally rely heavily on raw 2D axis-aligned views and often do not use even familiar tools such as de-noising filters, cutting planes, and maximum intensity projections. Depending on the case, a radiologist could spent significant time scrolling back and forth for understanding the relationship between lesions and duct, when a typical span of the pancreas can be 150-200 axial slices. In contrast, the systems and methods described herein provide 3D visualizations that improve the diagnosis process effectiveness and provide additional data points to assist in decision making. The following exemplary workflow can be carried out by a radiologist using the systems and methods described herein: (1) overview a case and inspect the lesions using 2D axis-aligned views and provided segmentation outlines; (2) visualize the lesion and its internal features in 3D to further understand the lesion morphological structure; (3) study the lesion and duct relationship in 3D, CPR view, and duct-centric re-sectioning views; and (4) confirm own diagnosis and characterization with the automatic classification results. Radiologists' accuracy using the VP systems and methods described herein will improve substantially from the current 67-70%

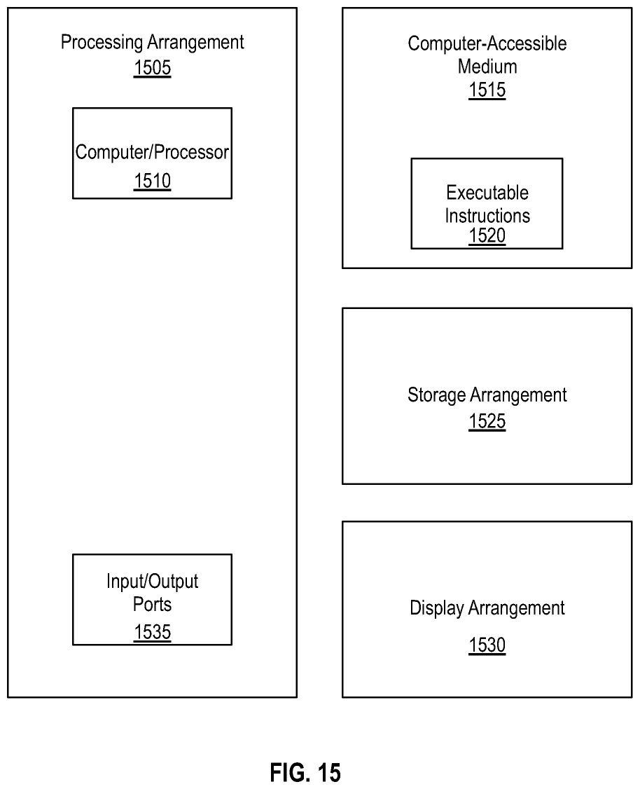

FIG. 15 shows a simplified block diagram of an exemplary embodiment of a system according to the present disclosure. For example, exemplary procedures in accordance with the present disclosure described herein (including the segmentation module, extraction module, classification module, and visualization module) can be performed by a processing arrangement and/or a computing arrangement (e.g., computer hardware arrangement) 1505. Such processing/computing arrangement 1505 can be, for example entirely or a part of, or include, but not limited to, a computer/processor 1510 that can include, for example one or more microprocessors, and use instructions stored on a computer-accessible medium (e.g., RAM, ROM, hard drive, or other storage device).

As shown in FIG. 15, for example a computer-accessible medium 1515 (e.g., as described herein above, a storage device such as a hard disk, floppy disk, memory stick, CD-ROM, RAM, ROM, etc., or a collection thereof) can be provided (e.g., in communication with the processing arrangement 1505). The computer-accessible medium 1515 can contain executable instructions 1520 thereon. In addition or alternatively, a storage arrangement 1525 can be provided separately from the computer-accessible medium 1515, which can provide the instructions to the processing arrangement 1505 so as to configure the processing arrangement to execute certain exemplary procedures, processes, and methods, as described herein above, for example.

Further, the exemplary processing arrangement 1505 can be provided with or include an input/output ports 1535, which can include, for example a wired network, a wireless network, the internet, an intranet, a data collection probe, a sensor, etc. As shown in FIG. 15, the exemplary processing arrangement 1505 can be in communication with an exemplary display arrangement 1530, which, according to certain exemplary embodiments of the present disclosure, can be a touch-screen configured for inputting information to the processing arrangement in addition to outputting information from the processing arrangement, for example. Further, the exemplary display arrangement 1530 and/or a storage arrangement 1525 can be used to display and/or store data in a user-accessible format and/or user-readable format.

It will be appreciated that in order to practice the methods of the embodiments as described above, it is not necessary that the processors and/or the memories be physically located in the same geographical place. That is, each of the processors and the memories used in exemplary embodiments of the present disclosure can be located in geographically distinct locations and connected so as to communicate in any suitable manner. Additionally, it is appreciated that each of the processor and/or the memory can be composed of different physical pieces of equipment. Accordingly, it is not necessary that the processor be one single piece of equipment in one location and that the memory be another single piece of equipment in another location. That is, it is contemplated that the processor can be two or more pieces of equipment in two or more different physical locations. The two distinct pieces of equipment can be connected in any suitable manner. Additionally, the memory can include two or more portions of memory in two or more physical locations.

As described above, a set of instructions is used in the processing of various embodiments of the present disclosure. The servers and personal computing devices described above can include software or computer programs stored in the memory (e.g., non-transitory computer readable medium containing program code instructions executed by the processor) for executing the methods described herein. The set of instructions can be in the form of a program or software or app. The software can be in the form of system software or application software, for example. The software might also be in the form of a collection of separate programs, a program module within a larger program, or a portion of a program module, for example. The software used might also include modular programming in the form of object oriented programming. The software tells the processor what to do with the data being processed.

Further, it will be appreciated that the instructions or set of instructions used in the implementation and operation of the present disclosure can be in a suitable form such that the processor can read the instructions. For example, the instructions that form a program can be in the form of a suitable programming language, which is converted to machine language or object code to allow the processor or processors to read the instructions. That is, written lines of programming code or source code, in a particular programming language, are converted to machine language using a compiler, assembler or interpreter. The machine language is binary coded machine instructions that are specific to a particular type of processor, i.e., to a particular type of computer, for example. Any suitable programming language can be used in accordance with the various embodiments of the present disclosure. For example, the programming language used can include assembly language, Ada, APL, Basic, C, C++, COBOL, dBase, Forth, Fortran, Java, Modula-2, Pascal, Prolog, REXX, Visual Basic, and/or JavaScript and others. Further, it is not necessary that a single type of instructions or single programming language be utilized in conjunction with the operation of the system and method of the present disclosure. Rather, any number of different programming languages can be utilized as is necessary or desirable.

Also, the instructions and/or data used in the practice of various embodiments of the present disclosure can utilize any compression or encryption technique or algorithm, as can be desired. An encryption module might be used to encrypt data. Further, files or other data can be decrypted using a suitable decryption module, for example.

The software, hardware and services described herein can be provided utilizing one or more cloud service models, such as Software-as-a-Service (SaaS), Platform-as-a-Service (PaaS), and Infrastructure-as-a-Service (IaaS), and/or using one or more deployment models such as public cloud, private cloud, hybrid cloud, and/or community cloud models.

In the system and method according to exemplary embodiments of the present disclosure, a variety of “user interfaces” can be utilized to allow a user to interface with the personal computing devices. As used herein, a user interface can include any hardware, software, or combination of hardware and software used by the processor that allows a user to interact with the processor of the communication device. A user interface can be in the form of a dialogue screen provided by an app, for example. A user interface can also include any of touch screen, keyboard, voice reader, voice recognizer, dialogue screen, menu box, list, checkbox, toggle switch, a pushbutton, a virtual environment (e.g., Virtual Machine (VM)/cloud), or any other device that allows a user to receive information regarding the operation of the processor as it processes a set of instructions and/or provide the processor with information. Accordingly, the user interface can be any system that provides communication between a user and a processor. The information provided by the user to the processor through the user interface can be in the form of a command, a selection of data, or some other input, for example.

Although the exemplary embodiments of the present disclosure have been described herein in the context of a particular implementation in a particular environment for a particular purpose, those skilled in the art will recognize that its usefulness is not limited thereto and that the embodiments of the present disclosure can be beneficially implemented in other related environments for similar purposes.

What is claimed is:

1. A method for virtual pancreatography, comprising:
receiving radiological images;
receiving at least one of patient demographic and clinical data;
performing automatic segmentation of the radiological images to generate at least one of a pancreas segment, a duct segment, and a lesion segment;
determining at least one of a pancreas and a duct centerline based on the at least one of the pancreas segment, the duct segment, and the lesion segment;
performing automatic lesion classification based on the radiological images, the at least one of demographic and clinical data, the at least one of the pancreas segment, the duct segment, and the lesion segment and the at least one of a pancreas and a duct centerline to determine a lesion classification; and
generating and displaying a three dimensional rendition based on the at least one of the pancreas segment, the duct segment, and the lesion segment, the at least one of a pancreas and a duct centerline and lesion classification on a display device.

2. The method of claim 1, further comprising performing a semi-automatic extraction of the at least one of the pancreas segment, the duct segment, and the lesion segment.

3. The method of claim 2, wherein the semi-automatic extraction is implemented with a multi-scale vesselness filter using an adjustable threshold parameter.

4. The method of claim 3, wherein the at least one of the pancreas segment, the duct segment, and the lesion segment, are passed through a duct extraction window to generate a primary pancreatic duct image.

5. The method of claim 1, wherein the performing automatic segmentation of the radiological images includes dividing the radiological images into a plurality of sub-volumes, detecting whether each of the plurality of sub-volumes includes a target structure for segmentation, and predicting a set of voxels that constitutes the target structure.

6. The method of claim 5, wherein the target structure is one of a healthy tissue or a lesion.

7. The method of claim 6, wherein the performing automatic segmentation of the radiological images further includes pre-training a model with subsamples of the sub-volumes extracted from the center of the target structure of the sub-volume.

8. The method of claim 1, wherein the determining at least one of a pancreas and a duct centerline is performed by modeling a curve that passes substantially through primary duct components.

9. The method of claim 1, wherein the determining at least one of a pancreas and a duct centerline is performed by determining centerlines for each of a plurality of connected components, and connecting consecutive components using a shortest penalized path.

10. The method of claim 1, wherein the performing automatic lesion classification is performed using a CAD algorithm comprising a probabilistic random forest (RF) classifier based on the at least one of demographic and clinical data and a convolutional neural network (CNN) based on the radiological images.

11. The method of claim 10, wherein classification probabilities are generated based on a bayesian combination of the RF and CNN

12. The method of claim 1, wherein the generating and displaying a three dimensional rendition includes generating and displaying a three dimensional visualization of the at least one of the pancreas segment, the duct segment, and lesion segment.

13. The method of claim 12, wherein the visualization is enhanced through a Hessian-based objectness filter.

14. The method of claim 13, further comprising: modifying one of an opacity and an offset slider, and adjusting one of a global opacity and a negative/positive offset of the visualization.

15. A system for virtual pancreatography (VP), comprising:
a segmentation module configured perform automatic segmentation of received radiological images to generate at least one of a pancreas segment, a duct segment, and a lesion segment;
a centerline module configured to determine at least one of a pancreas and a duct centerline based on at least one of the pancreas segment, the duct segment, and the lesion segment;
a curved planar reformation (CPR) module configured to perform CPR of the at least one of the pancreas segment, the duct segment, and the lesion segment and generate a CPR rendition;
a classification module configured to perform automatic lesion classification based on at least one of received demographic and clinical data, the radiologic images, the at least one of a pancreas segment, a duct segment, and a lesion segment; and
a visualization module configured to generate and display a three dimensional rendition based on the at least one of the pancreas segment, the duct segment, and the lesion segment, the at least one of a pancreas and a duct centerline and lesion classification on a display device, and the CPR rendition.

16. The system of claim 15, further comprising:
an extraction module configured to perform a semi-automatic extraction of the at least one of the pancreas segment, the duct segment, and the lesion segment.

17. The system of claim 15, wherein the segmentation module is further configured to divide the radiological images into a plurality of sub-volumes, detect whether each of the plurality of sub-volumes includes a target structure for segmentation, and predict a set of voxels that constitutes the target structure.

18. The system of claim 17, wherein the performing automatic segmentation of the radiological images further includes pre-training a model with subsamples of the sub-volumes extracted from the center of the target structure of the sub-volume.

19. The system of claim 15, wherein the classification module is further configured to perform automatic lesion classification using a CAD algorithm comprising a probabilistic random forest (RF) classifier based on the at least one demographic and clinical data and a convolutional neural network (CNN) based on the radiological images.

20. A non-transitory computer-accessible medium having stored thereon computer-executable instructions, wherein the computer arrangement comprises a processor, and wherein, upon execution of the instructions, the computer arrangement is configured to perform procedures comprising:
receiving radiological images;
receiving at least one of patient demographic and clinical data;
performing automatic segmentation of the radiological images to generate at least one of a pancreas segment, a duct segment, and a lesion segment;
determining at least one of a pancreas and a duct centerline based on the at least one of the pancreas segment, the duct segment, and the lesion segment;
performing automatic lesion classification based on the radiological images, the at least one of demographic and clinical data, the at least one of the pancreas segment, the duct segment, and the lesion segment and the at least one of a pancreas and a duct centerline to determine a lesion classification; and
generating and displaying a three dimensional rendition based on the at least one of the pancreas segment, the duct segment, and the lesion segment, the at least one of a pancreas and a duct centerline and lesion classification on a display device.
